(19) United States
(12) Patent Application Publication
(10) Pub. No.: US 2022/0204936 A1
(43) Pub. Date: Jun. 30, 2022

(54) NON-FIBROUS FILM AND CELL SHEET

(71) Applicant: Industrial Technology Research Institute, Hsinchu (TW)

(72) Inventors: Yu-Bing LIOU, Hsinchu City (TW); Chih-Ching LIAO, Xinpu Township (TW); Hsin-Yi HSU, Taoyuan City (TW); Ying-Wen SHEN, Zhunan Township (TW); Yun-Chung TENG, Kaohsiung City (TW); Hsin-Hsin SHEN, Zhudong Township (TW); Yi-Chen CHEN, Hsinchu City (TW)

(73) Assignee: Industrial Technology Research Institute, Hsinchu (TW)

(21) Appl. No.: 17/475,794

(22) Filed: Sep. 15, 2021

(30) Foreign Application Priority Data
Dec. 31, 2020 (TW) 109147086

(51) Int. Cl. C12N 5/077; C08J 5/18

(52) U.S. Cl. CPC C12N 5/0656; C08J 5/18; C12N 2533/54; C08J 2467/04; C12N 2533/40; C08J 2389/00

(57) ABSTRACT

A non-fibrous film of which the composition includes a collagen and a polyester polymer is provided. A content of the polyester polymer in the non-fibrous film is 1-60 wt %. Moreover, the non-fibrous film has a swelling rate of 1-200 μm/hour or a swelling proportion per unit time of 0.1-2%/hour in an aqueous liquid.

### CROSS REFERENCE TO RELATED APPLICATION

The present application is based on, and claims priority from, Taiwan Application Serial Number 109147086, filed on Dec. 31, 2020, the disclosure of which is hereby incorporated by reference herein in its entirety.

### TECHNICAL FIELD

The present disclosure relates to a non-fibrous film and a cell sheet.

### BACKGROUND

The primary purpose of regenerative medicine cell therapy is to replace, repair, improve or regenerate various tissues and organs of the body. In traditional regenerative medicine, a cell suspension or its combination with a cell complex carrier scaffold is usually used to deliver cells to the affected area. Although traditional regenerative cell therapy have greatly promoted the development of regenerative medicine, the harvest of cell suspensions often requires trypsin to suspend the cells, and this step usually damaged the surface proteins of the cells. Once the surface proteins of the cells are damaged, cell function is affected. When cell surface proteins are disrupted, the intercellular and extracellular matrix (ECM) interactions are reduced. These intercellular interactions and the production of extracellular matrix are essential to maintain tissue function. The use of trypsin to harvest cell suspensions will have a negative effect on the adhesion and proliferation of cells, resulting in uneven distribution of cells at the implantation site or reduced repair efficiency after injection of cell suspensions, and even causing adverse reactions such as inflammation at the implantation site.

Since the production of cell sheet does not require protein hydrolysis enzyme treatment, and the interactions between cells and the structure of cell sheet are well preserved on cell sheet, cell sheet for cell therapy tissue regeneration has become an emerging cell delivery method in recent years. The cell sheet technology has a higher cell concentration and more uniform cell distribution, and thus increasing the tissue regeneration capacity. In addition, the cells are stimulated by the culture medium and substrate in the cell sheet production, and can be induced to form cells with different characteristics. This phenomenon is particularly evident in mesenchymal stem cells (MSCs), which have the function of replacing or restoring damaged tissues. Guiding stem cells toward differentiated cells with specific function at the early stage of cellular sheet construction and maintaining these cellular characteristics is the most challenging cell culture procedure at present.

Currently, two main approaches are used to induce cell differentiation, one is to induce cell differentiation by chemical stimulation, and the other is to induce cell differentiation by physical mechanical stimulation or by stimulation of the surface structure of the substrate to which the cells are attached. Since chemical stimulation is difficult to control due to the concentration gradient and action time of the formula used, the biomimetic environment made with physical characteristics of mechanical stimulation and the surface structure of the substrate has better control effects and has a wider range of applications.

Mechanical stimulation and substrate surface structural stimulation have a decisive effect on the differentiation of mesenchymal stem cells. Therefore, in order to optimize and guide the pre-differentiation of stem cells to specific tissue cell characteristics, there is a strong need for a novel substrate that can provide both mechanical stimulation and substrate surface structural stimulation to induce cell differentiation.

### SUMMARY

The present disclosure provides a non-fibrous film of which the composition comprises: a collagen; and a polyester polymer. A content of the polyester polymer in the non-fibrous film is 1-60 wt %. Moreover, the non-fibrous film has a swelling rate of 1-200 μm/hour or a swelling proportion per unit time of 0.1-2%/hour in an aqueous liquid.

The present disclosure also provides another non-fibrous film which is prepared by a method. The method comprises: (a) preparing a mixture solution; and (b) drying the mixture solution to form a film. A solute of the mixture solution comprises: a collagen and a polyester polymer, wherein a content of the polyester polymer in the non-fibrous film is 1-60 wt %. A solvent of the mixture solution comprises a perfluorocarbon solvent. Moreover, the non-fibrous film has a swelling rate of 1-200 μm/hour or a swelling proportion per unit time of 0.1-2%/hour in an aqueous liquid.

The present disclosure also provides a cell sheet comprising any non-fibrous film mentioned above; and at least one cell layer on the non-fibrous film. The cell layer is composed of a plurality of cells, and the plurality of cells is arranged in the same direction.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1A shows the results of Fourier transform infrared spectroscopy (FTIR) analysis of the non-fibrous film prepared by Preparation example 1 (100 wt % collagen (hexafluoroisopropanol used as a solvent in the preparation thereof)) and the non-fibrous film prepared by Comparative preparation example 1 (100 wt % collagen (an acidic aqueous solution used as a solvent in the preparation thereof));

FIG. 1B shows the results of fitting the Fourier transform infrared spectra of the non-fibrous film prepared by Preparation example 1 (100 wt % collagen (hexafluoroisopropanol used as a solvent in the preparation thereof)) and the non-fibrous film prepared by Comparative preparation example 1 (100 wt % collagen (an acidic aqueous solution used as a solvent in the preparation thereof));

FIG. 2 shows a scanning electron microscopy photograph of the non-fibrous film prepared by Preparation example 8 (50 wt % collagen and 50 wt % polycarprolactone).

FIG. 3 shows a scanning electron microscopy photograph of the electrospun film (100 wt % of collagen) prepared by Comparative preparation example 3;

FIG. 4A shows the film diameters of the non-fibrous film prepared in Preparation example 1 (100 wt % collagen) determined in RO pure water, 0.9% saline, 10 mM Tri-HCl buffer, Dulbecco's phosphate buffered saline (DPBS), Dulbecco's Modified Eagle Medium (DMEM) or Dulbecco's Modified Eagle Medium (DMEM) containing 10% fetal bovine serum (FBS) (cell culture medium) at different time points in the film swelling test;

FIG. 4B shows the film diameters of the non-fibrous film prepared by Preparation example 2 (90 wt % collagen and 10 wt % polycarprolactone) determined in RO pure water, 0.9% saline, 10 mM Tri-HCl buffer, Dulbecco's phosphate buffered saline (DPBS), Dulbecco's Modified Eagle Medium (DMEM) or Dulbecco's Modified Eagle Medium (DMEM) containing 10% fetal bovine serum (FBS) (cell culture medium) at different time points in the film swelling test;

FIG. 4C shows the film diameters of the non-fibrous film prepared by Preparation example 7 (70 wt % collagen and 30 wt % polycarprolactone) prepared in Preparation example 7 determined in RO pure water, 0.9% saline, 10 mM Tri-HCl buffer, Dulbecco's phosphate buffered saline (DPBS), Dulbecco's Modified Eagle Medium (DMEM) or Dulbecco's Modified Eagle Medium (DMEM) containing 10% fetal bovine serum (FBS) (cell culture medium) at different time points in the film swelling test;

FIG. 4D shows the film diameters of the non-fibrous film prepared by Preparation example 8 (non-fibrous film: 50 wt % collagen and 50 wt % polycarprolactone) determined in RO pure water, 0.9% saline, 10 mM Tri-HCl buffer, Dulbecco's phosphate buffered saline (DPBS), Dulbecco's Modified Eagle Medium (DMEM) or Dulbecco's Modified Eagle Medium (DMEM) containing 10% fetal bovine serum (FBS) (cell culture medium) at different time points in the film swelling test;

FIG. 5A shows the film diameters of the non-fibrous film prepared by Preparation example 1 (100 wt % collagen), the non-fibrous film prepared by Preparation example 2 (90 wt % collagen and 10 wt % polycarprolactone), the non-fibrous film prepared by Preparation example 7 (70 wt % collagen and 30 wt % polycarprolactone) prepared in Preparation example 7, and the non-fibrous film prepared by Preparation example 8 (non-fibrous film: 50 wt % collagen and 50 wt % polycarprolactone) in Dulbecco's Modified Eagle Medium (DMEM) containing 10% fetal bovine serum (FBS) (cell culture medium) determined at different time points in the film swelling test;

FIG. 5B shows the swelling rates of the non-fibrous film prepared by Preparation example 1 (100 wt % collagen), the non-fibrous film prepared by Preparation example 2 (90 wt % collagen and 10 wt % polycarprolactone), the non-fibrous film prepared by Preparation example 7 (70 wt % collagen and 30 wt % polycarprolactone) prepared in Preparation example 7, and the non-fibrous film prepared by Preparation example 8 (non-fibrous film: 50 wt % collagen and 50 wt % polycarprolactone) in Dulbecco's Modified Eagle Medium (DMEM) containing 10% fetal bovine serum (FBS) (cell culture medium) determined at different time points in the film swelling test;

FIG. 6 shows the swelling rates and swelling proportion per unit time of the non-fibrous film prepared by Preparation example 4 (80 wt % collagen and 20 wt % polycarprolactone, crosslinking for 1.5 hours), the non-fibrous film prepared by Preparation example 5 (80 wt % collagen and 20 wt % polycarprolactone, crosslinking for 2 hours) and the non-fibrous film prepared by Preparation example 6 (80 wt % collagen and 20 wt % polycarprolactone, crosslinking for 3 hours) in DMEM solution containing 10% fetal bovine serums;

FIG. 7 shows the film diameters of the non-fibrous film prepared in preparation example 9 (90 wt % collagen and 10 wt % polylactic acid) determined in a PBS buffer at different time points in the film swelling test;

FIG. 8 shows the film area of the electrospun film prepared in Comparative preparation example 3 (100 wt % collagen) and the electrospun film prepared in Comparative preparation example 4 (50 wt % collagen and 50 wt % polycarprolactone) determined in PBS buffer at different time points in the film swelling test;

FIG. 9A shows photographs of the growth status of cells on the non-fibrous film prepared in prepared in Preparation example 3 (80 wt % collagen and 20 wt % polycarprolactone) and cells on the culture plate on Day 1, 3, 7 and 14 in the cell culture test;

FIG. 9B shows photographs of the growth status of cells on the non-fibrous film prepared in prepared in Preparation example 3 (80 wt % collagen and 20 wt % polycarprolactone) and cells on the culture plate on Day 3 in the cell culture test; and

FIG. 10 shows a photograph of the growth status of cells on the non-fibrous film prepared in prepared in Comparative preparation example 2 (90 wt % collagen and 10 wt % polycarprolactone (an acidic aqueous solution used as a solvent)) on Day 6 in the cell culture test.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure may provide a non-fibrous film which may be a biodegradable film, and this film may have dynamic changes in size over time in an aqueous liquid. There is no particular limitation for the aqueous liquid mentioned above, as long as it does not result in adverse effects on the film. Examples of the aqueous fluids may include, but are not limited to, water, a buffer, and a cell culture medium.

The non-fibrous film of the present disclosure mentioned above may have cell-bearing capabilities and may be applied to cell culture. When the non-fibrous film of the present disclosure is applied to cell culture, since its size change dynamically in a cell culture medium, during cell culture, it can provide the cells which are attached thereto to grow with a physical mechanical force, such as a tensile stress, and a stimulus of structural changes on the surface to which the cells are attached, without the need of providing an additional mechanical and/or chemical stimulus to the film and/or cells, to allow the cells which are attached thereto to grow in the same direction and to promote cell differentiation.

The non-fibrous film of the present disclosure mentioned above may be formed by a method, and the method comprises, but it is not limited to the following steps.

First, a mixture solution is prepared.

The concentration of the solute in the prepared mixture solution is not particularly limited and can be adjusted as needed, as long as it does not adversely affect the formation of the film. For example, the concentration of solute in the mixture solution prepared can be adjusted according to the environment at the time of preparing the mixture solution (e.g., temperature, humidity, pressure of the environment), the type of solvent used, the environment at the time of subsequent film formation (e.g., temperature, humidity, pressure, etc.), and the method for film formation to be used, but is not limited thereto. For example, the concentration of the solute in the mixture solution may be about 0.5-20 wt %, such as about 0.5 wt %, about 0.8 wt %, about 1 wt %, about 3 wt %, about 5 wt %, 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, but it is not limited thereto.

Furthermore, the solute in the mixture solution mentioned above may comprise, but is not limited to a collagen and a polyester polymer.

The content of the polyester polymer in the solute mentioned above may be about 1-60 wt %, such as about 1-55 wt %, about 1-50 wt %, about 10-50 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, but it is not limited thereto.

Moreover, in the solute mentioned above, a weight ratio of the collagen to the polyester polymer may be about 0.6-99:1, such as about 95:5, about 90:10, about 85:15, about 80:20, about 75:25, about 70:30, about 65:35, about 60:40, about 55:45, about 50:50, about 45:55, about 40:60, but it is not limited thereto.

In one embodiment, the solute mentioned above may consist of collagen and polyester polymer, and the weight ratio of the collagen to the polyester polymer may be about 0.6-99:1, such as about 95:5, about 90:10, about 85:15, about 80:20, about 75:25, about 70:30, about 65:35, about 60:40, about 55:45, about 50:50, about 45:55, about 40:60, but it is not limited thereto.

The collagen in the solute mentioned above has no particular limitations, and may include any type of collagen. Example of the collagen mentioned above may comprise at least one of the following: type I collagen, type II collagen and type III collagen, but it is not limited thereto. In one embodiment, the collagen mentioned above is type I collagen.

The molecular weight of the polyester polymer in the solute mentioned above may depend on the need, and has no particular limitation. For example, the molecular weight of the polyester polymer mentioned above may be about 50,000-1,500,000 Da, such as medium molecular weight (about 50,000-200,000 Da), high molecular weight (about 800,000-1,500,000 Da), about 50,000 Da, about 60,000 Da, about 70,000 Da, about 80,000 Da, about 90,000 Da, about 100,000 Da, about 150,000 Da, about 200,000 Da, about 250,000 Da, about 300,000 Da, about 400,000, about 500,000 Da, about 800,000 Da, about 1,000,000 Da, about 1,500,000 Da, but it is not limited thereto. In one embodiment, the molecular weight of the polyester polymer may be about 80,000 Da.

Furthermore, the intrinsic viscosity of the polyester polymer in the solute mentioned above may also depend on the need, and has no particular limitation. For example, the intrinsic viscosity of the polyester polymer mentioned above may be about 0.15-7 dl/g, such as about 0.15-0.3 dl/g, about 0.35-0.45 dl/g, about 0.8-1.2 dl/g, about 1.3-1.6 dl/g, about 1.6-2.4 dl/g, about 2.0-2.8 dl/g, about 3.5-5.5 dl/g. In one embodiment, the intrinsic viscosity of the polyester polymer mentioned above may be about 4.5 dl/g.

In addition, the type of the polyester polymer in the solute mentioned above also has no particular limitation, as long as it is biodegradable. Example of the polyester polymer may comprise, but is not limited to, polycarprolactone (PCL), polylactic acid (PLA), polyglycolic acid (PGA), polyhydroxybutyrate (PHB), polyhydroxyvalerate (PHV), or any combination thereof. In one embodiment, the polyester polymer in the solute mentioned above may be polycarprolactone. In another embodiment, the polyester polymer in the solute mentioned above may be polylactic acid.

Moreover, a solvent of the mixture solution may comprise a perfluorocarbon solvent, but it is not limited thereto. Examples of the perfluorocarbon solvent mentioned above may include, but are not limited to, hexafluoroisopropanol, hexafluoroacetone, 1,1,1-trichlorotrifluoroacetone, trifluoroacetic acid, perfluoroperhydrophenanthrene, perfluoro(methylcyclohexane), or any combination thereof. The solvent mentioned above may be used as a co-solvent for the collagen and the polyester polymer in the solute mentioned above, and can allow the collagen and the polyester polymer in the mixture solution mentioned above uniformly distributed. In addition, the solvent mentioned above can make collagen exist in the mixture solution mentioned above and the formed film in both the native form and the denatured form at the same time. In one example, the solvent of the mixture solution is hexafluoroisopropanol.

Next, after the mixture solution mentioned above is prepared, the mixture solution mentioned above is dried to form a film to obtain the non-fibrous film of the present disclosure.

The manner of drying the mixture solution mentioned above to form a film has no particular limitation, as long as the mixture solution mentioned above is able to form a film. In one embodiment, the mixture solution mentioned above can be poured into a mold and then dried to form a film. In another embodiment, the mixture solution mentioned above can be poured on a flat plate, scraped by a scraper, and then dried to form a film.

In one embodiment, the obtained non-fibrous film may be a porous film. In this embodiment, the content of the polyester polymer in the above-mentioned solute mentioned above may be about 45-60 wt %, such as about 45 wt %, about 50 wt %, about 55 wt %, about 60 wt %, but it is not limited thereto. In this embodiment, the pore size of the pores of the porous film may be about 1-50 μm, such as about 5-50 μm, about 10-50 μm, about 15-50 about 1-45 about 1-40 about 1-30 about 1 about 5 about 10 about 15 about 20 about 25 about 30 about 35 about 40 about 45 about 50 but it is not limited thereto.

In the foregoing embodiment where the obtained non-fibrous film may be a porous film, in the method for preparing the non-fibrous film of the present disclosure mentioned above, the solute of the mixture solution prepared may consist of collagen and polyester polymer. The polyester polymer may include at least one of the following: polycarprolactone, polylactic acid, polyglycolic acid, polyhydroxybutyrate, polyhydroxyvalerate, etc., but is not limited thereto. In one specific embodiment, when the obtained non-fibrous film is a porous film, in the method of preparing the non-fibrous film of the present disclosure mentioned above, the solute of the mixture solution consists of the collagen and the polyester polymer, and the polyester polymer is polycarprolactone. Moreover, in this specific embodiment, the molecular weight of the polycarprolactone may be about 50,000-200,000 Da, such as about 80,000 Da, but is not limited thereto.

In another embodiment, the obtained non-fibrous film may be a non-porous film. In this embodiment, the content of the polyester polymer in the solute mentioned above may be about 1 wt % or more but less than 45 wt %, such as about 1-40 wt %, about 5-40 wt %, about 10-40 wt %, about 15-35 wt %, about 20-30 wt %, about 1 wt %, about 3 wt %, about 5 wt %, about 10 wt %, about 15 wt %, about 20 wt %, about 25 wt %, about 30 wt %, about 35 wt %, about 40 wt %, about 44 wt %, but not limited thereto.

In the foregoing embodiment where the obtained non-fibrous film may be a non-porous film, in the method for preparing the non-fibrous film of the present disclosure mentioned above, the solute of the mixture solution prepared can be made of collagen and polyester. The polyester polymer may include at least one of the following: polycarprolactone, polylactic acid, polyglycolic acid, polyhydroxybutyrate, polyhydroxyvalerate, etc., but is not limited thereto. In one specific embodiment, when the obtained non-fibrous film is a non-porous film, in the method of preparing the non-fibrous film of the present disclosure mentioned above, the solute of the prepared mixture solution consists of the collagen and the polyester polymer, and the polyester polymer is polylactic acid. In this specific embodiment, the intrinsic viscosity of the polylactic acid may be about 0.15-7.0 dl/g, such as about 4.5 dl/g, but is not limited thereto.

Furthermore, in one embodiment, the method for preparing the non-fibrous film of the present disclosure mentioned above, in addition to the step of preparing a mixture solution and the step of drying the foregoing mixture solution to form a film mentioned above, may also further comprise after drying the foregoing mixture solution to form a film, performing a cross-linking treatment on the formed film to form a cross-linked non-fibrous film.

The time for the foregoing cross-linking treatment to the film has no particular limitation, and can be adjusted as needed. For example, the treatment time for the cross-linking treatment to the film can be adjusted based on the environment at the time of performing the cross-linking treatment (e.g., temperature, humidity, pressure of the environment), the constituent components and/or content of the components of the film, the cross-linking method or the cross-linking agent used, the desired film strength, the use of the film, etc., but it is not limited thereto. For example, treatment time for the foregoing cross-linking treatment to the film may be about 0.1-10 hours, such as about 0.1 hour, about 0.5 hour, about 1 hour, about 1.5 hours, about 2 hours, about 2.5 hours, about 3 hours, about 5 hours, about 6 hours, about 8 hours, about 10 hours, etc., but not limited thereto.

In addition, the cross-linking treatment to the film mentioned above may be performed with a cross-linking agent, but it is not limited thereto. Examples of the cross-linking agent mentioned above may include formaldehyde, glutaraldehyde, glyoxal, malondialdehyde, succinyl dialdehyde, phthalaldehyde, dialdehyde starch, polyacrolein, polymethacrolein, and any combination thereof, but they are not limited thereto.

In one embodiment, the cross-linking treatment to the film mentioned above may be performed with formaldehyde, such as 10% formaldehyde vapor, and the treatment time for the cross-linking treatment to the film may be about 0.1-10 hours, such as about 0.1 hour, about 0.5 hour, about 1 hour, about 1.5 hours, about 2 hours, about 2.5 hours, about 3 hours, about 5 hours, about 6 hours, about 8 hours, about 10 hours, but it is not limited thereto.

According to the foregoing, it is known that in the method for preparing the non-fibrous film of the present disclosure mentioned above, the solvent of the mixture solution can make collagen exist in the mixture solution and the formed film in both the native form and the denatured form at the same time. Since both natural collagen and denatured collagen exist in the formed film at the same time, when the non-fibrous film is in contact with a liquid, the natural collagen and denatured collagen molecules in the film due to hydration force have a competitive effect with each other in terms of force with the polyester polymer to allow the film has physical property of dynamic swelling or stretching.

Namely, as mentioned above, the non-fibrous film of the present disclosure prepared by any method described above may have a dynamic change in size over time in a liquid.

The degree of dynamic changes in size of the non-fibrous film of the present disclosure in a liquid, such as the degree of swelling or stretching, may be adjusted by selecting different constituent components and/or contents of the components of the film for the solute of the mixture solution, or by adjusting the time for the cross-linking treatment when the film undergoes a cross-linking treatment, in the method for preparing the non-fibrous film of the present disclosure mentioned above, but it is not limited thereto. For example, the degree of dynamic changes in size of the non-fibrous film of the present disclosure in a liquid can be adjusted by adjusting the weight ratio of the collagen to the polyester polymer in the solute of the mixture solution, by adjusting the type and molecular weight/intrinsic viscosity of the polyester polymer in the solute of the mixture solution, and/or by adjusting the time of the cross-linking treatment when the film undergoes a cross-linking treatment.

In one embodiment, the non-fibrous film of the present disclosure prepared by any method described above may have a swelling rate of about 1-200 μm/hour in an aqueous liquid, such as about 5-200 μm/hour, about 10-200 μm/hour, about 10-150 μm/hour, about 1 μm/hour, about 2 μm/hour, about 5 μm/hour, about 10 μm/hour, about 11 μm/hour, about 100 μm/hour, about 103 μm/hour, about 110 μm/hour, about 120 μm/hour, about 130 μm/hour, about 140 μm/hour, about 148 μm/hour, about 150 μm/hour, about 200 μm/hour, but not limited thereto. In another embodiment, the non-fibrous film of the present disclosure prepared by any method described above may have a swelling proportion per unit time of about 0.1-2%/hour in an aqueous liquid, such as about 0.1-1.5%/hour, about 0.1-1%/hour, about 0.1-0.5%/hour, about 0.1-0.2%/hour, about 0.2-2%/hour, about 0.5-2%/hour, about 0.1-2%/hour, about 0.15-2%/hour, about 0.1%/Hour, about 0.2%/hour, about 0.5%/hour, about 1%/hour, about 1.5%/hour, about 2%/hour, but not limited thereto.

Furthermore, based on the foregoing content, the present disclosure may also provide another non-fibrous film. This non-fibrous film may also be a biodegradable film and may have dynamic changes in size over time in an aqueous liquid. Also, the non-fibrous film may have cell-bearing capabilities and may be applied to cell culture, and when it is applied to cell culture, since it can continuously provide the cells which are attached thereto to grow with a physical mechanical force, such as a tensile stress, it can allow the cells which are attached thereto to grow in the same direction and to promote cell differentiation. The aqueous liquid has no particular limitation, as long as it has no negative impact on the film. Examples of aqueous fluids may include, but are not limited to, water, a buffer, and a culture medium.

The non-fibrous film mentioned above may have a swelling rate of about 1-200 μm/hour in an aqueous liquid, such as about 5-200 μm/hour, about 10-200 μm/hour, about 10-150 μm/hour, about 1 μm/hour, about 2 μm/hour, about 5 μm/hour, about 10 μm/hour, about 11 μμm/hour, about 100 μm/hour, about 103 μm/hour, about 110μ, m/hour, about 120 μm/hour, about 130 μm/hour, about 140 μm/hour, about 148 μm/hour, about 150 μm/hour, about 200 μm/hour, but it is not limited thereto.

Alternatively, the non-fibrous film mentioned above may have a swelling proportion per unit time of about 0.1-2%/hour in an aqueous liquid, such as about 0.1-1.5%/hour, about 0.1-1%/hour, about 0.1-0.5%/hour, about 0.1-0.2%/hour, about 0.2-2%/hour, about 0.5-2%/hour, about 0.1-2%/hour, about 0.15-2%/hour, about 0.1%/hour, about 0.2%/hour, about 0.5%/hour, about 1%/hour, about 1.5%/hour, about 2%/hour, but it is not limited thereto.

The composition of the above non-fibrous film may include, but is not limited to, a collagen and a polyester polymer. Furthermore, the content of the polyester polymer in the non-fibrous film mentioned above may be about 1-60 wt %, such as about 1-55 wt %, about 1-50 wt %, about 10-50 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, but not limited thereto. In one embodiment, the collagen in the non-fibrous film mentioned above may have both natural form and denatured form at the same time.

Moreover, in the non-fibrous film mentioned above, a weight ratio of the collagen to the polyester polymer may be about 0.6-99:1, such as about 95:5, about 90:10, about 85:15, about 80:20, about 75:25, about 70:30, about 65:35, about 60:40, about 55:45, about 50:50, about 45:55, about 40:60, but it is not limited thereto. In one embodiment, the composition of the non-fibrous film mentioned above may consists of collagen and polyester polymer, and the weight ratio of the collagen to the polyester polymer may be about 0.6 to 99:1, such as about 95:5, about 90:10, about 85:15, about 80:20, about 75:25, about 70:30, about 65:35, about 60:40, about 55:45, about 50:50, about 45:55, about 40:60, but it is not limited thereto.

The collagen in the composition of the non-fibrous film mentioned above has no particular limitation, and may include any type of collagen. Example of the collagen mentioned above may comprise, but is not limited to, at least one of the following: type I collagen, type II collagen and type III collagen. In one embodiment, the collagen mentioned above is type I collagen.

The molecular weight of the polyester polymer in the composition of the non-fibrous film mentioned above may depend on the need, and has no particular limitation. For example, the molecular weight of the polyester polymer mentioned above may be about 50,000-1,500,000 Da, such as medium molecular weight (about 50,000-200,000 Da), high molecular weight (about 800,000-1,500,000 Da), about 50,000 Da, about 60,000 Da, about 70,000 Da, about 80,000 Da, about 90,000 Da, about 100,000 Da, about 150,000 Da, about 200,000 Da, about 250,000 Da, about 300,000 Da, about 400,000, about 500,000 Da, about 800,000 Da, about 1,000,000 Da, about 1,500,000 Da, but it is not limited thereto. In one embodiment, the molecular weight of the polyester polymer may be about 80,000 Da.

Furthermore, the intrinsic viscosity of the polyester polymer mentioned above may be about 0.15-7 dl/g, such as about 0.15-0.3 dl/g, about 0.35-0.45 dl/g, about 0.8-1.2 dl/g, about 1.3-1.6 dl/g, about 1.6-2.4 dl/g, about 2.0-2.8 dl/g, about 3.5-5.5 dl/g. In one embodiment, the intrinsic viscosity of the polyester polymer mentioned above may be about 4.5 dl/g.

In addition, the type of polyester polymer in the composition of the non-fibrous film mentioned above also has no particular limitation, as long as it is biodegradable. Example of the polyester polymer may comprise, but is not limited to polycarprolactone (PCL), polylactic acid (PLA), polyglycolic acid (PGA), polyhydroxybutyrate (PHB), polyhydroxyvalerate (PHV), and any combination thereof. In one embodiment, the polyester polymer in in the composition of the non-fibrous film mentioned above may be polycarprolactone. In another embodiment, the polyester polymer in the composition of the non-fibrous film mentioned above may be polylactic acid.

In one embodiment, the non-fibrous film mentioned above may be a porous film. In this embodiment, the content of the polyester polymer in the non-fibrous film mentioned above may be about 45-60 wt %, such as about 45 wt %, 50 wt %, about 55 wt %, about 60 wt %, but it is not limited thereto. Also in this embodiment, the pore size of the pores of the porous film may be about 1-50 μm, such as about 5-50 μm, about 10-50 μm, about 15-50 μm, about 1-45 μm, about 1-40 μm, about 1-30 μm, about 1 μm, about 5 μm, about 10 μm, about 15 μm, about 20 μm, about 25 μm, about 30 μm, about 35 μm, about 40 μm, about 45 μm, about 50 μm, but it is not limited thereto.

In the foregoing embodiment in which the non-fibrous film may be a porous film, the composition of the non-fibrous film may consist of the collagen and the polyester polymer, and the polyester polymer may include at least one of the following: polycarprolactone, polylactic acid, polyglycolic acid, polyhydroxybutyrate, polyhydroxypentanoate, etc., but is not limited thereto. In one specific embodiment, when the non-fibrous film is the porous film mentioned above, the composition of the non-fibrous film consists of the collagen and the polyester polymer, and the polyester polymer is polycarprolactone. Moreover, in this specific embodiment, the molecular weight of the polycarprolactone may be about 50,000-200,000 Da, such as about 80,000 Da, but is not limited thereto.

In another embodiment, the non-fibrous film mentioned above may be a non-porous film. In this embodiment, the content of the polyester polymer in the non-fibrous film mentioned above may be about 1 wt % or more but less than 45 wt %, such as about 1-40 wt %, about 5-40 wt %, about 10-40 wt %, about 15-35 wt %, about 20-30 wt %, about 1 wt %, about 3 wt %, about 5 wt %, about 10 wt %, about 15 wt %, about 20 wt %, about 25 wt %, about 30 wt %, about 35 wt %, about 40 wt %, about 44 wt %, etc., but not limited to.

In the foregoing embodiment in which the non-fibrous film may be a non-porous film, the composition of the non-fibrous film may comprise the collagen and the polyester-like polymer, and the polyester polymer may include at least one of the following: polycarprolactone, polylactic acid, polyglycolic acid, polyhydroxybutyrate, poly hydroxypentanoate, etc., but it is not limited thereto. In one specific embodiment, when the non-fibrous film mentioned above is a non-porous film, the composition of the non-fibrous film consists of the collagen and the polyester-like polymer, and the polyester polymer is polylactic acid. Furthermore, in this specific embodiment, the intrinsic viscosity of the polylactic acid may be about 0.15-7.0 dl/g, such as about 4.5 dl/g, but is not limited thereto.

Furthermore, in one embodiment, the non-fibrous film mentioned above may be an uncrosslinked film, or may be a crosslinked film.

The crosslinked film mentioned above may be subjected to a cross-linking treatment. The treatment time for the foregoing cross-linking treatment to the film has no particular limitation, and can be adjusted as needed. For example, the treatment time for the cross-linking treatment to the film can be adjusted based on the environment at the time of performing the cross-linking treatment (e.g., temperature, humidity, pressure of the environment), the constituent components and/or content of the components of the film, the cross-linking method or the cross-linking agent used, the desired film strength, the use of the film, etc., but it is not limited thereto. For example, treatment time for the foregoing cross-linking treatment to the film may be about 0.1-10 hours, such as about 0.1 hour, about 0.5 hour, about 1 hour, about 1.5 hours, about 2 hours, about 2.5 hours, about 3 hours, about 5 hours, about 6 hours, about 8 hours, about 10 hours, but it is not limited thereto.

In addition, the cross-linking treatment mentioned above can be performed with a cross-linking agent, but is not limited thereto. Examples of the cross-linking agent mentioned above may include formaldehyde, glutaraldehyde, glyoxal, malondialdehyde, succinyl dialdehyde, phthalaldehyde, dialdehyde starch, polyacrolein, polymethacrolein, and any combination thereof, but they are not limited thereto.

In one embodiment, the cross-linking treatment to the film mentioned above may be performed with formaldehyde, such as 10% formaldehyde vapor, and the treatment time for the cross-linking treatment to the film may be about 0.1-10 hours, such as about 0.1 hour, about 0.5 hour, about 1 hour, about 1.5 hours, about 2 hours, about 2.5 hours, about 3 hours, about 5 hours, about 6 hours, about 8 hours, about 10 hours, but it is not limited thereto.

In addition, the present disclosure may also provide a method for culturing cells, which may include, but is not limited to, culturing cells on any non-fibrous film of the present disclosure mentioned above in a culture medium.

Since any non-fibrous film of the present disclosure mentioned above may have cell-bearing capabilities, and its size changes dynamically in a culture medium, during cell culture, it can continuously provide the cells which are attached thereon to grow with a physical mechanical force, such as a tensile stress, and a stimulus of structural changes of the surface to which the cells are attached, without the need of providing an additional mechanical and/or chemical stimulus to the film and/or cells, to allow the cells which are attached thereto to grow in the same direction and/or to promote cell differentiation.

The cells used in the method for culturing cells of the present disclosure can be selected as needed and has no particular limitation. For example, the type of cell can be selected according to the desired application field, but it is not limited thereto. In one embodiment, the cells need to be used for tissue regeneration in a living body, and thus cells with differentiation ability can be used in the method of culturing cells of the present disclosure mentioned above. Examples of cells with differentiation ability may include, but are not limited to, fibroblast, myoblasts, epithelial cells, endothelial cells, progenitor cells, tenocytes, stem cells, mesenchymal stem cells, bone marrow stem cells, and adipose derived stem cells.

In the method for culturing cells of the present disclosure, the culture medium used for culturing the cells can be selected as needed and has no particular limitation. For example, the selection can be made according to the cell to be cultured, the state to be achieved by the cell, but it is not limited thereto.

In addition, in the method for culturing cells of the present disclosure, the time for culturing cells can be adjusted as needed, and has no particular limitation. For example, the time for culturing the cells can be adjusted according to the type of cells, the growth status of the cells, the environment in which the cells are cultured (such as the temperature and humidity of the environment), the culture medium used, and so on. In one embodiment, the time for culturing the cells may be about 1-28 days, such as about 1 day, about 1.5 days, about 2 days, about 3 days, about 5 days, about 6 days, about 7 days, about 10 days, about 14 days, about 21 days, about 28 days, but it is not limited thereto.

Furthermore, in the method for culturing cells of the present disclosure, the temperature of culturing cells can be adjusted as needed, and has no particular limitation. For example, the temperature of the cultured cells can be adjusted according to the type of cells, the growth status of the cells, the culture medium used, and so on. In one embodiment, the temperature for culturing the cells may be about 30-40° C., such as about 30° C., about 32° C., about 35° C., about 36° C., about 37° C., about 37.5° C., about 38° C., about 39° C., about 40° C., but it is not limited thereto.

Furthermore, in the method for culturing cells of the present disclosure, the cells mentioned above may grow into at least one layer of cells on a non-fibrous film. In other words, in the method of culturing cells of the present disclosure, the cells mentioned above may grow into a single layer of cells or grow into multiple layers of cells on the non-fibrous film.

In one embodiment, the method for culturing cells of the present disclosure may further comprise, after the cells mentioned above growing into at least one layer of cells on the non-fibrous film, culturing cells which are different type from that of the cells mentioned above in a culture medium which is the same as or different from the foregoing culture medium, on this non-fibrous film with at least one layer of cells, to allow different cells be able to grow into at least one layer of cells. This step can be repeated as needed, and each time this step is repeated, the selected cells are different from the previous one.

Regarding the selection and culture conditions of different cells, please refer to the foregoing paragraphs with regard to cell selection, culture medium selection, culturing time and culturing temperature, etc., and thus they are not repeated herein.

Moreover, similarly, the present disclosure may also provide a method for forming a cell sheet, which may comprise culturing cells in a culture medium on any non-fibrous film of the present disclosure mentioned above to allow the cells grow in the same direction on any non-fibrous film of the present disclosure mentioned above and form at least one layer of cells to form a cell sheet, but it is not limited thereto.

Since any non-fibrous film of the present disclosure mentioned above may have cell-bearing capabilities, and its size changes dynamically in a culture medium, during cell culture, it can continuously provide the cells which are attached thereon to grow with a physical mechanical force, such as a tensile stress, and a stimulus of structural changes of the surface to which the cells are attached, without the need of providing an additional mechanical and/or chemical stimulus to the film and/or cells, to allow the cells which are attached thereto to grow in the same direction and/or to promote cell differentiation to obtain a cell sheet which has at least one layer of cells of a plurality of cells arranged in the same direction.

In one embodiment, the method for forming a cell sheet of the present disclosure may further comprise, after the cells mentioned above growing into at least one layer of cells on the non-fibrous film, culturing cells which are different type from that of the cells mentioned above in a culture medium which is the same as or different from the foregoing culture medium, on this non-fibrous film with at least one layer of cells, to allow different cells be able to grow into at least one layer of cells. This step can be repeated as needed, and each time this step is repeated, the selected cells are different from the previous one.

Regarding the selection and culture conditions of different cells, please refer to the descriptions for cell selection, culture medium selection, culturing time and culturing temperature, etc. in the foregoing paragraphs with regard to the method for culturing cells of the present disclosure, and thus they are not repeated herein.

Furthermore, based on the above, the present disclosure may also provide a cell sheet, which may include, but is not limited to, any non-fibrous film of the present disclosure mentioned above, and at least one layer of cells on any non-fibrous film of the present disclosure mentioned above, wherein the cell layer may be composed of a plurality of cells, and the plurality of cells may be arranged in the same direction. In one embodiment, the cells in the cell sheet of the present disclosure may have better differentiation ability.

In one embodiment, the cell sheet of the present disclosure may have multiple layers of cell layers, and the cells in each layer of cells may be the same or different.

The cells in the cell sheet of the present disclosure mentioned above may be selected as needed, and has no particular limitation. For example, the type of cell can be selected according to the desired field to which the cell sheet applied, but it is not limited thereto. In one embodiment, the cell sheet need to be used for tissue regeneration in a living body, and thus cells with differentiation ability can be used in the cell sheet of the present disclosure mentioned above. Examples of cells with differentiation ability may include, but are not limited to, fibroblast, myoblasts, epithelial cells, endothelial cells, progenitor cells, tenocytes, stem cells, mesenchymal stem cells, bone marrow stem cells, and adipose derived stem cells.

### EXAMPLES

### Example 1: Preparation of Film

### 1. Preparation Example 1

Non-fibrous film: 100 wt % of collagen

Type I collagen and hexafluoroisopropanol (HFP) as a solvent were uniformly mixed with to prepare a 10% (w/w) collagen solution.

15 g of this collagen solution was poured into a Teflon mold (a bottom dimension of which was 7 cm*7 cm) and placed in a fume hood to dry to form a film.

After drying to form the film, the film was removed from the mold and cross-linked with 10% formaldehyde for 1 hour. Afterwards, a cross-linked non-fibrous film with a collagen content of 100 wt % was obtained.

### 2. Preparation Example 2

Non-fibrous film: 90 wt % collagen and 10 wt % polycarprolactone (PCL)

1.574 g of type I collagen and 0.17 g of polycarprolactone (Mw: 80,000 Da) were added to 17.9 g of hexafluoroisopropanol which was used as a solvent and uniformly mixed to prepare a mixture solution.

The mixture solution is poured into a Teflon mold (a bottom dimension of which was 7 cm*7 cm) and placed in a fume hood to dry to form a film.

After drying to form the film, the film was removed from the mold and cross-linked with 10% formaldehyde for 1 hour. Afterwards, a cross-linked non-fibrous film with a collagen content of 90 wt % was obtained.

### 3. Preparation Example 3

Non-fibrous film: 80 wt % collagen and 20 wt % polycarprolactone

1.36 g of type I collagen and 0.34 g of polycarprolactone (Mw: 80,000 Da) were added to 16 g of hexafluoroisopropanol which was used as a solvent and uniformly mixed to prepare a mixture solution.

The mixture solution was poured into a Teflon mold (a bottom dimension of which was 7 cm*7 cm) and placed in a fume hood to dry to form a film.

After drying to form the film, the film was removed from the mold and cross-linked with 10% formaldehyde for 1 hour. Afterwards, a cross-linked non-fibrous film with a collagen content of 80 wt % was obtained.

### 4. Preparation Example 4

Non-fibrous film: 80 wt % collagen and 20 wt % polycarprolactone, crosslinking for 1.5 hours

Type I collagen, polycarprolactone (Mw: 80,000 Da) and hexafluoroisopropanol as solvent were uniformly mixed to prepare a mixture solution with a solute concentration of 10% (w/w), wherein the weight ratio of type I collagen to polycarprolactone was 8:2.

The mixture solution was poured into a Teflon mold (a bottom dimension of which was 7 cm*7 cm) and placed in a fume hood to dry to form a film.

After drying to form the film, the film was removed from the mold and cross-linked with 10% formaldehyde for 1.5 hours. Afterwards, a cross-linked non-fibrous film (cross-linking time 1.5 hours) with a collagen content of 80 wt % was obtained.

### 5. Preparation Example 5

Non-fibrous films: 80 wt % collagen and 20 wt % polycarprolactone, crosslinking for 2 hours.

The preparation conditions and steps were the same as those in Preparation example 4, except that the crosslinking time was adjusted to 2 hours.

### 6. Preparation Example 6

Non-fibrous films: 80 wt % collagen and 20 wt % polycarprolactone, crosslinking for 3 hours

The preparation conditions and steps were the same as those in Preparation example 4, except that the crosslinking time was adjusted to 3 hours.

### 7. Preparation Example 7

Non-fibrous film: 70 wt % collagen and 30 wt % polycarprolactone

2.38 g of type I collagen and 1.08 g of polycarprolactone (Mw: 80,000 Da) were added to 21.76 g of hexafluoroisopropanol which was used as a solvent and uniformly mixed to prepare a mixture solution.

The mixture solution was poured into a Teflon mold (a bottom dimension of which was 7 cm*7 cm) and placed in a fume hood to dry to form a film.

After drying to form the film, the film was removed from the mold and cross-linked with 10% formaldehyde for 1 hour. Afterwards, a cross-linked non-fibrous film with a collagen content of 70 wt % was obtained.

### 8. Preparation Example 8

Non-fibrous film: 50 wt % collagen and 50 wt % polycarprolactone

2.34 g of type I collagen and 2.34 g of polycarprolactone (Mw: 80,000 Da) were added to 46.3 g of hexafluoroisopropanol which was used as a solvent and uniformly mixed to prepare a mixture solution.

The mixture solution was poured into a Teflon mold (a bottom dimension of which was 7 cm*7 cm) and placed in a fume hood to dry to form a film.

After drying to form the film, the film was removed from the mold and cross-linked with 10% formaldehyde for 1 hour. Afterwards, a cross-linked non-fibrous film with a collagen content of 50 wt % was obtained.

### 9. Preparation Example 9

Non-fibrous film: 90 wt % collagen with 10 wt % polylactic acid (PLA)

4.61 g of type I collagen and 0.512 g of poly (D-lactic acid) (PDLA) (4.5 dl/g) were added to 68.89 g of hexafluoroisopropanol which was used as a solvent and uniformly mixed to prepare a mixture solution.

69.15 g of this mixture solution was poured into a Teflon mold (a bottom dimension of which was 20 cm*20 cm) and placed in a fume hood to dry to form a film.

After drying to form the film, the film was removed from the mold and cross-linked with 10% formaldehyde for 1 hour. Afterwards, a cross-linked non-fibrous film with a collagen content of 90 wt % was obtained.

### 10. Preparation Example 10

Non-fibrous film: 70 wt % collagen with 30 wt % polylactic acid

3.32 g of type I collagen and 1.42 g of poly (D-lactic acid) (PDLA) (4.5 dl/g) were added to 68.89 g of hexafluoroisopropanol which was used as a solvent and uniformly mixed to prepare a mixture solution.

69.15 g of this mixture solution was poured into a Teflon mold (a bottom dimension of which was 20 cm*20 cm) and placed in a fume hood to dry to form a film.

After drying to form the film, the film was removed from the mold and cross-linked with 10% formaldehyde for 1 hour. Afterwards, a cross-linked non-fibrous film with a collagen content of 70 wt % was obtained.

### 11. Comparative Preparation Example 1

Non-fibrous film: 100 wt % collagen (acidic aqueous solution as a solvent)

Type I collagen and an acidic aqueous solution (pH 3) as solvent were uniformly mixed to prepare a 1% (w/w) collagen solution.

The collagen solution was poured into a Teflon mold (a bottom dimension of which was 13 cm*13 cm) and placed in a fume hood to dry to form a film.

After drying to form the film, the film was removed from the mold and cross-linked with 10% formaldehyde for 1 hour. Afterwards, a cross-linked non-fibrous film with a collagen content of 100 wt % was obtained.

### 12. Comparative Preparation Example 2

Non-fibrous type film: 90 wt % collagen with 10 wt % polycarprolactone (acidic aqueous solution as a solvent)

Type I collagen and polyvinylpyrrolidone (1(30) were added to an acidic aqueous solution (pH3) which was used as a solvent and uniformly mixed to prepare a mixture solution (concentration of type I collagen was 0.9% (w/w) and concentration of polyvinylpyrrolidone was 0.1 (w/w).

67 g of this mixture solution was poured into a Teflon mold (a bottom dimension of which was 13 cm*13 cm) and placed in a fume hood to dry to form a film.

After drying to form the film, the film was removed from the mold and cross-linked with 10% formaldehyde for 1 hour. Afterwards, a cross-linked non-fibrous film with a collagen content of 90 wt % was obtained.

### 13. Comparative Preparation Example 3

Electrospun film: 100 wt % of collagen

Type I collagen and hexafluoroisopropanol as a solvent were uniformly mixed to prepare a 10% (w/w) collagen solution.

The collagen solution was used as a raw material to electrospun by an electrospinning device (needle 25-26G; solution flow rate 0.15 mL/hour; voltage 5-6 kV) to form an electrospun film. The electrospun film manufactured was cross-linked with 10% formaldehyde for 1 hour. After that, a cross-linked electrospun film with a collagen content of 100 wt % was obtained.

### 14. Comparative Preparation Example 4

Electrospun films: 50 wt % collagen and 50 wt % polycarprolactone

Type I collagen, polycarprolactone (Mw: 80,000 Da) and hexafluoroisopropanol as a solvent were uniformly mixed to prepare a mixture solution with a solute concentration of 10% (w/w), wherein the weight ratio of type I collagen to polycarprolactone was 1:1.

The collagen solution was used as a raw material to electrospun by an electrospinning device (needle 25-26G; solution flow rate 0.15 mL/hour; voltage 5-6 kV) to form an electrospun film. The electrospun film manufactured was cross-linked with 10% formaldehyde for 1 hour. After that, a cross-linked electrospun film with a collagen content of 50 wt % was obtained.

### Example 2

Fourier-Transform Infrared Spectroscopy (FTIR)

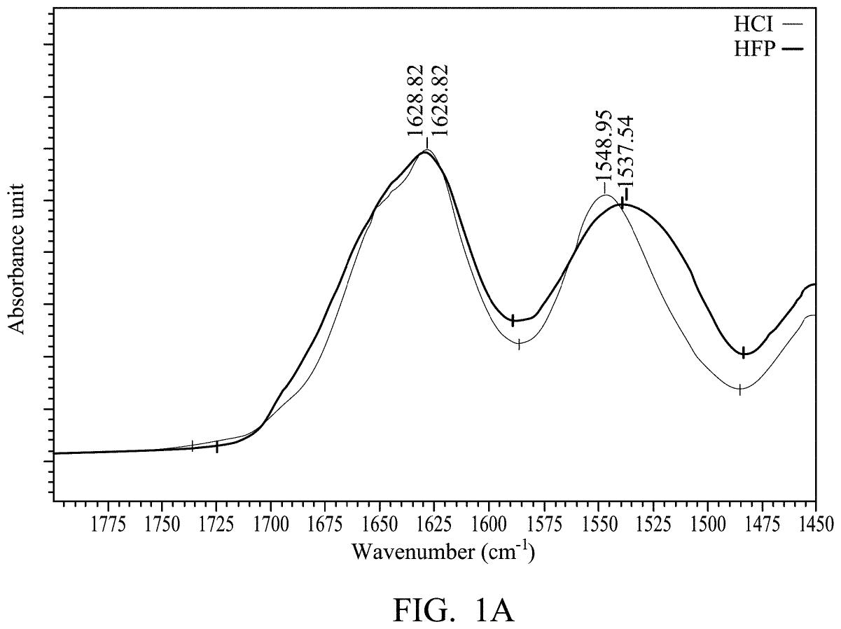

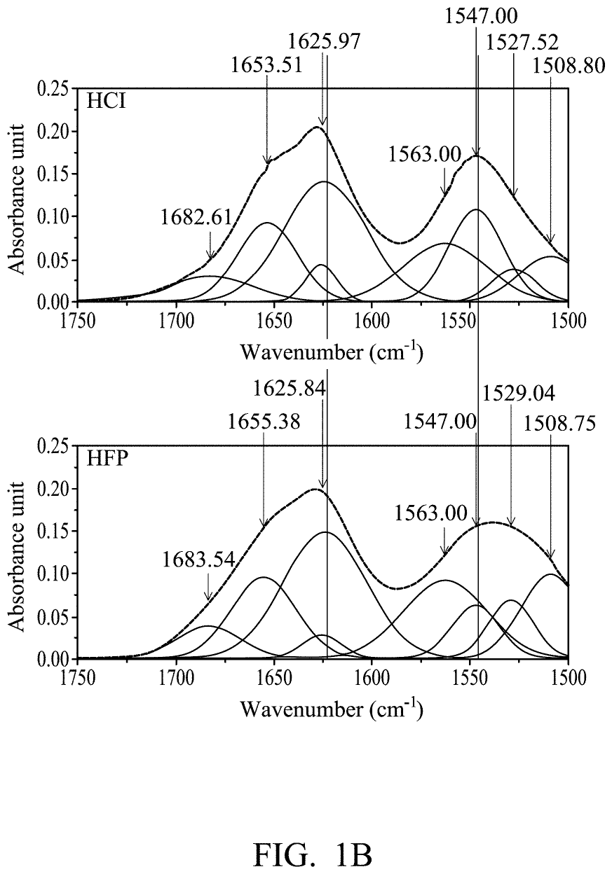

The non-fibrous film prepared by Preparation example 1 (100 wt % collagen (hexafluoroisopropanol was used as a solvent in the preparation thereof)) and the non-fibrous film prepared by Comparative preparation example 1 (100 wt % collagen (an acidic aqueous solution was used as a solvent in the preparation thereof)) were subjected Fourier-transform infrared spectroscopy (FTIR) analysis. The results are shown in FIG. 1A.

Also, Tables 1 and 2 show the structures represented by the peaks of collagen in Fourier transform infrared spectroscopy (based on Olena S. Rabotyagova et al, “Collagen structural hierarchy and susceptibility to degradation. susceptibility to degradation by ultraviolet radiation” Materials Science and Engineering C 28 (2008), pp. 1420-1429).

TABLE 1
Assignment of the band components of amide I region

| Position | |
|---|---|
| (cm⁻¹) | Assignment |
| 1690 | Intermolecular association. Helixes of aggregated |
| | collagen-like peptides |
| | β-turn |
| 1675 | Collagen in triple helix, with contribution from |
| | alpha-helix and beta-turns |
| | (protein amide stretching vibration of C═O) |
| 1660 | Random chain contribution of the non-proline and |
| | non-hydroxyproline |
| 1646-1650 | Random coil conformation: imide residues |
| | (amide I in random coil) |
| | Left-handed 3-10 helix in denatured state |
| 1630 | Partial beta-sheet region |

TABLE 2
Assignment of the band components of amide II region

| Position | | |
|---|---|---|
| (cm⁻¹) | Assignment | Note |
| 1565 | Carboxyl groups | Side chains of D and E |
| | N—H bending vibration and C—N | |
| | stretching vibration | |
| 1549 | Amide II in triple helix | Collagen in triple helix |
| 1530 | Amide II in a random coil | Disordered structure due to |
| | | degeneration |
| 1515 | Tyrosine side chain | Transformation of F to Y |

According to Tables 1 and 2, it is known that the peak at 1549 cm⁻¹ represents the characteristics of a triple helix of collagen, the peak at 1530 cm⁻¹ represents the loose structure of denatured collagen, and the peak at 1646-1650 cm⁻¹ and the peak at 1660 cm⁻¹ represent the collagen structure appearance of random coil.

Therefore, based on the results of FIG. 1A and Tables 1 and 2, it is known that compared to the non-fibrous film prepared by Comparative preparation example 1 (100 wt % collagen (an acidic aqueous solution was used as a solvent in the preparation thereof)), amide II band for the non-fibrous film prepared by Preparation example 1 (100 wt % collagen (hexafluoroisopropanol was used as a solvent in the preparation thereof)) significantly shifts from peak at 1548.9 cm⁻¹, where it was originally located, to the peak at 1537.5 cm⁻¹, indicating a decrease in the triple helix structure and an increase in the denatured collagen structure.

Moreover, the Fourier transform infrared spectra of the non-fibrous film prepared by Preparation example 1 (100 wt % collagen (hexafluoroisopropanol was used as a solvent in the preparation thereof)) and the non-fibrous film prepared by Comparative preparation example 1 (100 wt % collagen (an acidic aqueous solution was used as a solvent in the preparation thereof)) shown in FIG. 1A were fitted and the result are shown in FIG. 1B and Table 3.

TABLE 3

| Non-fibrous film prepared by Comparative preparation | | | Non-fibrous film prepared by Preparation | | |
|---|---|---|---|---|---|
| example 1 (an acidic aqueous solution was used | | | example 1 (hexafluoroisopropanol was used | | |
| as a solvent in the preparation thereof) | | | as a solvent in the preparation thereof) | | |
| Peak (cm⁻¹) | Area | Ratio (%) | Peak (cm⁻¹) | Area | Ratio (%) |
| 1508.8 | 1.219 | 6.57 | 1508.75 | 1.905 | 10.63 |
| 1527.52 | 0.847 | 4.57 | 1529.04 | 1.374 | 7.67 |
| 1547 | 2.954 | 15.94 | 1547 | 1.329 | 7.42 |
| 1563 | 2.857 | 15.41 | 1563 | 3.431 | 19.15 |
| 1624.02 | 5.915 | 31.91 | 1624.12 | 5.549 | 30.98 |
| 1625.97 | 0.682 | 3.68 | 1625.84 | 0.4784 | 2.67 |
| 1653.51 | 2.785 | 15.02 | 1655.38 | 2.784 | 15.54 |
| 1682.61 | 1.277 | 6.89 | 1683.54 | 1.064 | 5.94 |

According to the results shown in FIG. 1B and Table 3 and Tables 1 and 2, it is known that compared to the non-fibrous film prepared by Comparative preparation example 1 (an acidic aqueous solution was used as a solvent in the preparation thereof), for the non-fibrous film prepared by Preparation example 1 (hexafluoroisopropanol was used as a solvent in the preparation thereof), area proportion of the peak at 1547 cm⁻¹ representing the characteristic of the collagen amide II triple helix reduces from about 15.94% to about 7.42%, the area proportion of the peak at 1527 cm⁻¹ representing the loose structure of collagen amide II after denaturation increase from about 4.57% to about 7.67%, and the area proportion of the peak at 1653 cm⁻¹ representing the collagen amide I random coil conformation increases from about 15.02% to about 15.54%.

Based on the foregoing, it is known that compared to the non-fibrous film prepared by Comparative preparation example 1 (an acidic aqueous solution was used as a solvent in the preparation thereof), the non-fibrous film prepared by Preparation example 1 (hexafluoroisopropanol was used as a solvent in the preparation thereof) has more denatured collagen, i.e., both natural collagen and denatured collagen are present in the non-fibrous film prepared by Preparation example 1 (hexafluoroisopropanol was used as a solvent in the preparation thereof) at the same time.

In other words, by using hexafluoroisopropanol as a solvent in the preparation of the collagen-containing film, the prepared film can have presence of both natural collagen and denatured collagen at the same time.

### Example 3

Scanning Electron Microscopy (SEM)

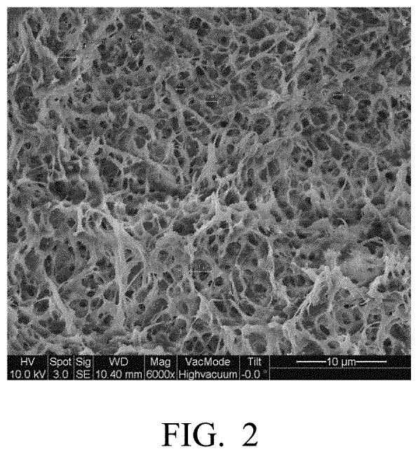

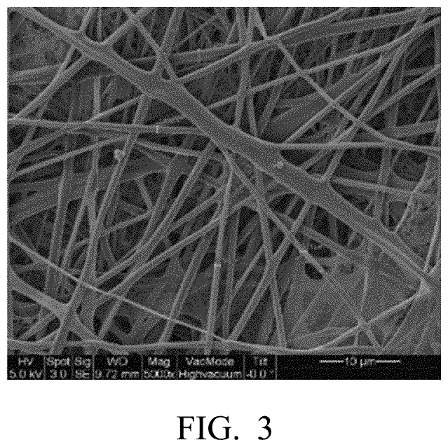

The non-fibrous film prepared by Preparation example 8 (50 wt % collagen and 50 wt % polycarprolactone) and the electrospun film (100 wt % collagen) prepared by Comparative preparation example 3 were analyzed by scanning electron microscope, and the results are shown in FIG. 2 and FIG. 3, respectively.

FIG. 2 shows a scanning electron microscopy photograph of the non-fibrous film prepared by Preparation example 8 (50 wt % collagen and 50 wt % polycarprolactone). According to FIG. 2, it is known that the non-fibrous film prepared by Preparation example 8 (50 wt % collagen and 50 wt % polycarprolactone) is a porous non-fibrous film with a pore size of about 1 μm.

FIG. 3 shows a scanning electron microscopy photograph of the electrospun film (100 wt % of collagen) prepared by Comparative preparation example 3. According to FIG. 3, it is known that the electrospun film (100 wt % collagen) prepared by Comparative example 3 is a fibrous film.

### Example 4

Film Swelling Test

The films prepared in the Preparation examples and the Comparative preparation examples were separately subjected to swelling tests.

1. Method

The film to be tested was cut by a circular cutter to obtain a circular film of 8 mm diameter.

The circular film was immersed in an aqueous solution (RO pure water, 0.9% saline, 10 mM Tri-HCl buffer, Dulbecco's phosphate buffered saline (DPBS), Dulbecco's Modified Eagle Medium (DMEM) or Dulbecco's Modified Eagle Medium (DMEM) containing 10% fetal bovine serum (FBS) (cell culture medium)).

When the film was immersed for 0 minute, 60 minutes, 24 hours, 72 hours, 7 days and 14 days, it was photographed by a microscope (NIKON model) at 0.75 magnification and the diameter thereof was measured.

The diameters for the film measured at the two time points were selected, and the swelling rate of the film could be calculated based on the following formula:

k=(y2−y1)/(x2−x1)

k is the swelling rate, x1 is the first time point, x2 is the second time point, y1 is the film diameter measured at the first time point, and y2 is the film diameter measured at the second time point.

2. Results

(1) With Regard to the Films Prepared in Preparation Example 1, 2, 3, 7 and 8

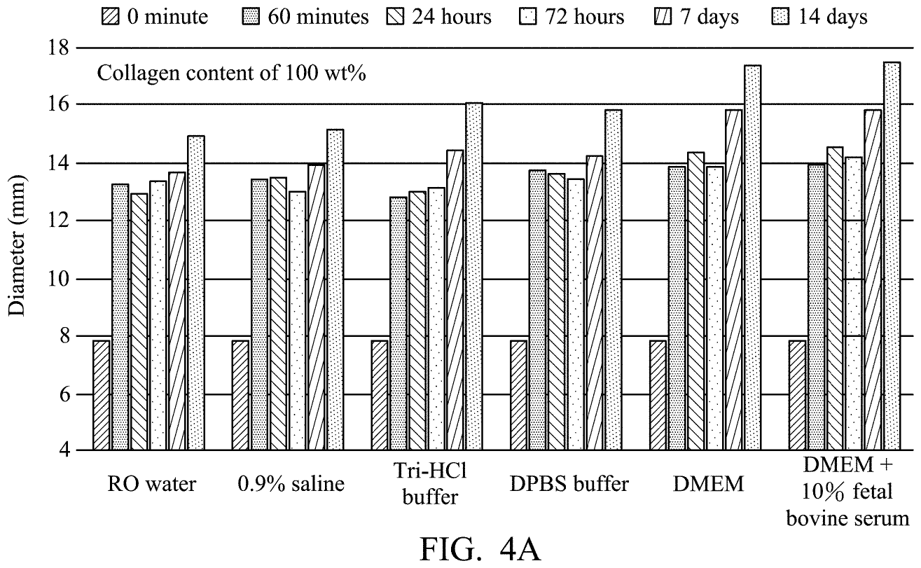

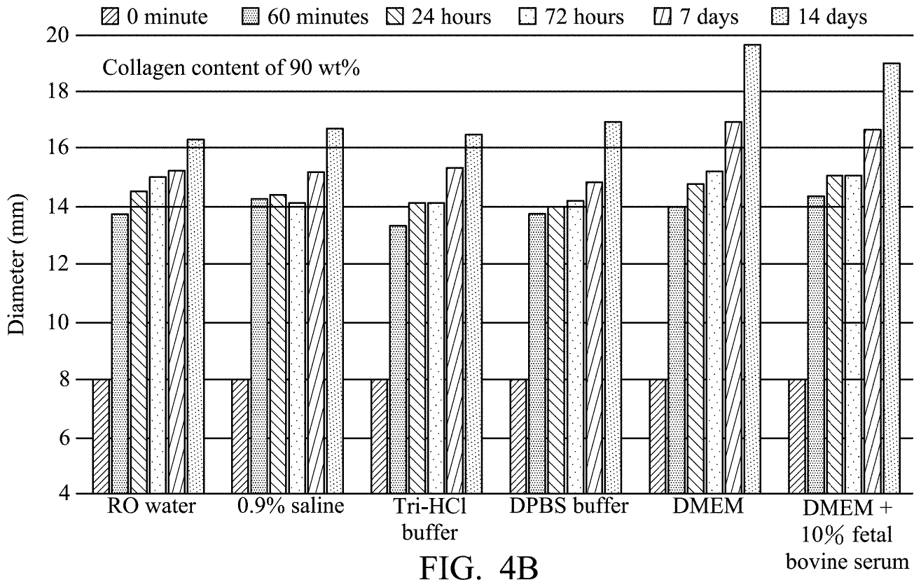

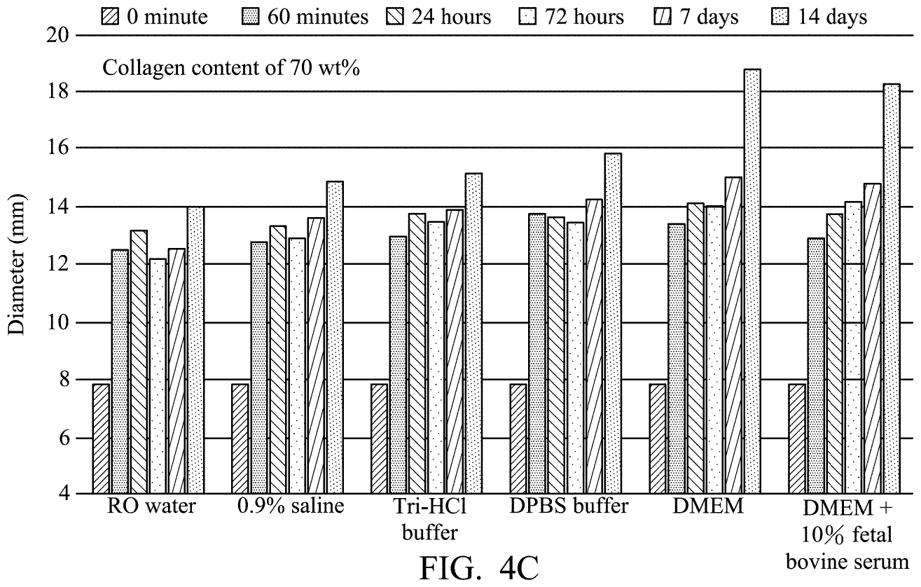

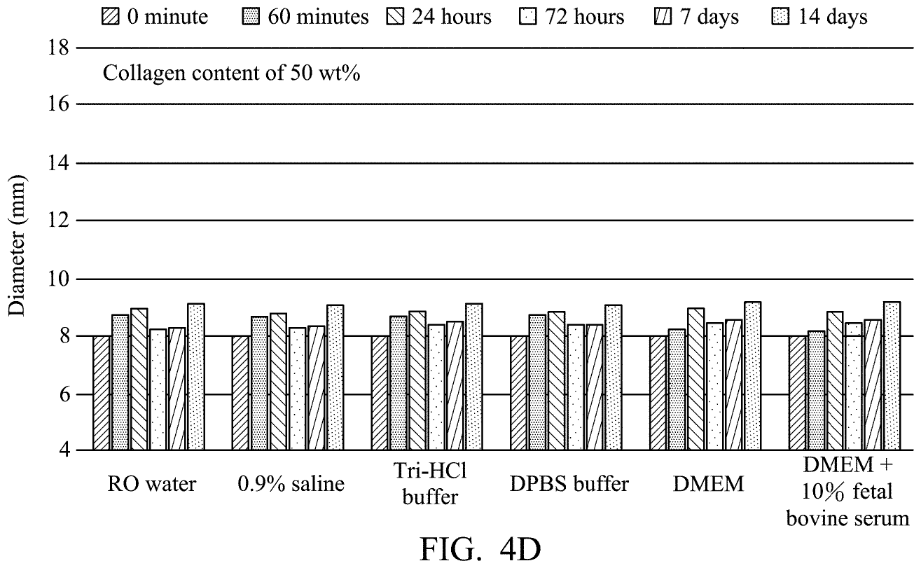

The film diameters of the non-fibrous film prepared by Preparation example 1 (100 wt % collagen), the non-fibrous film prepared by Preparation example 2 (90 wt % collagen and 10 wt % polycarprolactone), the non-fibrous film prepared by Preparation example 7 (70 wt % collagen and 30 wt % polycarprolactone) prepared in Preparation example 7, and the non-fibrous film prepared by Preparation example 8 (non-fibrous film: 50 wt % collagen and 50 wt % polycarprolactone) determined in different aqueous liquids at different time points are shown in FIG. 4A, FIG. 4B, FIG. 4C and FIG. 4D, respectively.

According to FIG. 4A, FIG. 4B, FIG. 4C and FIG. 4D, it is known that the sizes of each kind of the films in different aqueous liquids changes significantly with increasing time.

Based on the diameters of the films before the time point of 7 days, it is known that the continuous swelling effects of the films was more significant in the aqueous solutions containing amphoteric-ions (zwitterion), such as Tri-HCl buffer, DMEM solution and DMEM solution containing 10% fetal bovine serum (cell culture medium). It is assumed that the aqueous solutions containing amphiphilic ions have more significant hydrogen bonding effects among collagen proteins, which leads to better hydration and faster swelling rates for the films.

Moreover, the hydration effect of the film is particularly significant in DMEM solution and DMEM solution containing 10% fetal bovine serum (cell culture medium). Since DMEM solution and cell culture medium contain amino acid components in addition to amphoteric ions, which also increase the hydration of collagen films, the swelling rates for the films therein much greater than those in other aqueous solutions (RO pure water, saline, Tri-HCl buffer or phosphate buffer solution).

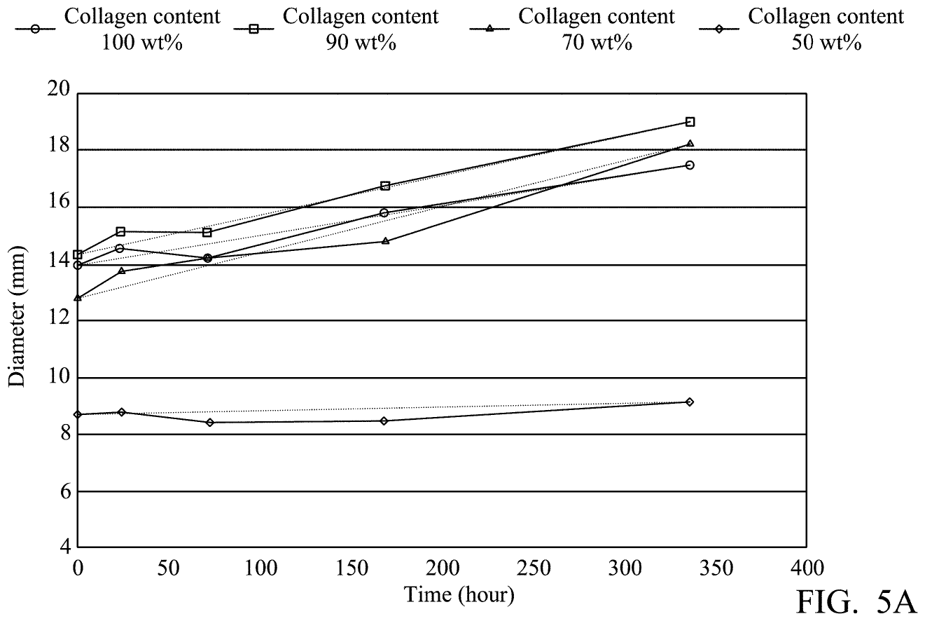

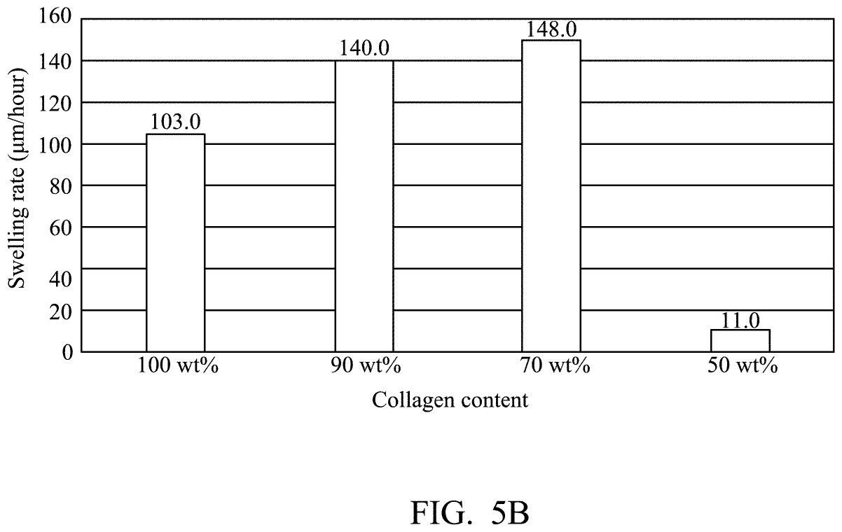

Furthermore, the film diameters of the non-fibrous film prepared by Preparation example 1 (100 wt % collagen), the non-fibrous film prepared by Preparation example 2 (90 wt % collagen and 10 wt % polycarprolactone), the non-fibrous film prepared by Preparation example 7 (70 wt % collagen and 30 wt % polycarprolactone) prepared in Preparation example 7, and the non-fibrous film prepared by Preparation example 8 (non-fibrous film: 50 wt % collagen and 50 wt % polycarprolactone) in DMEM solution containing 10% fetal bovine serum determined at different time points shown in FIG. 4A, FIG. 4B, FIG. 4C and FIG. 4D are integrated in FIG. 5A.

Moreover, the swelling rate of each film was calculated based on the diameter of each film in DMEM solution containing 10% fetal bovine serum determined at the time point 60 minutes and at the time point 14 days. The results are shown in FIG. 5B.

In addition, compared to at the time point 60 minutes, the swelling proportions of each film in each kind of aqueous solutions at the time point 7 days and the time point 14 days are calculated, respectively. The formula for calculating the swelling proportion is shown below.

Swelling proportion (%)=(diameter of film at the second time point−diameter of film at the first time point)/diameter of film at the first time point*100

In this experiment, the first time point is 60 minutes and the second time point is 7 days or 14 days.

The results are shown in Table 4.

TABLE 4
Compared to at the time point 60 minutes, the swelling proportions of each film in each kind
of aqueous solutions at the time point 7 days and the time point 14 days

| | | Swelling proportion (%) | | | | | |
|---|---|---|---|---|---|---|---|
| | | | | | | | DMEM solution |
| | | RO | 0.9% | Tri-HCl | | | containing |
| | | pure | physiological | buffer | Phosphate | | 10% fetal bovine |
| | Time | water | saline | solution | buffer | DMEM | serum |
| The film prepared | 7 | 3.16 | 3.00 | 12.39 | 3.46 | 13.99 | 13.86 |
| in Preparation | days | | | | | | |
| example 1 (100 | 14 | 12.32 | 12.69 | 25.61 | 14.94 | 25.18 | 25.85 |
| wt % collagen | days | | | | | | |
| | | RO | 0.9% | Tri-HCl | | | |
| | | pure | physiological | buffer | Phosphate | | Tri-HCl |
| | Time | water | saline | solution | buffer | DMEM | buffer solution |
| The film prepared | 7 | 11.01 | 6.17 | 15.03 | 7.65 | 20.78 | 16.18 |
| in Preparation | days | | | | | | |
| example 2 (90 | 14 | 18.52 | 16.48 | 23.50 | 22.29 | 40.03 | 31.82 |
| wt % collagen | days | | | | | | |
| and 10 wt % | | | | | | | |
| polycarprolactone | | | | | | | |
| | | | | | | | DMEM solution |
| | | RO | 0.9% | Tri-HCl | | | containing |
| | | pure | physiological | buffer | Phosphate | | 10% fetal bovine |
| | Time | water | saline | solution | buffer | DMEM | serum |
| The film prepared | 7 | 4.20 | 4.37 | 6.63 | 5.83 | 9.86 | 9.04 |
| in Preparation | days | | | | | | |
| example 3 (80 | 14 | 11.34 | 14.26 | 21.67 | 17.62 | 28.02 | 23.42 |
| wt % collagen | days | | | | | | |
| and 20 wt % | | | | | | | |
| polycarprolactone | | | | | | | |
| | | RO | 0.9% | Tri-HCl | | | |
| | | pure | physiological | buffer | Phosphate | | Tri-HCl |
| | Time | water | saline | solution | buffer | DMEM | buffer solution |
| The film prepared | 7 | 0.10 | 7.04 | 7.38 | 3.35 | 12.19 | 15.00 |
| in Preparation | days | | | | | | |
| example 7 (70 | 14 | 11.70 | 16.91 | 16.85 | 20.58 | 39.73 | 41.64 |
| wt % collagen | days | | | | | | |
| and 30 wt % | | | | | | | |
| polycarprolactone | | | | | | | |
| Preparation | 7 | −4.48 | −3.53 | −2.13 | −3.47 | 3.89 | 4.86 |
| example 8 (50 | days | | | | | | |
| wt % collagen | 14 | 4.60 | 4.64 | 4.65 | 3.97 | 11.29 | 12.34 |
| and 50 wt % | days | | | | | | |
| polycarprolactone | | | | | | | |

FIG. 5A, FIG. 5B and Table 4 show that the swelling proportions of the films containing 70-90 wt % collagen are greater than that of films containing 100 wt % collagen, and the swelling proportions of the films in each kind of aqueous liquids also increase with the increase in the content of the polyester polymer.

Accordingly, it is known that that the addition of the polyester polymer can impede the crosslinking among collagens and thus affect the intermolecular forces of collagen, resulting in an increase in the swelling rate of the film containing polyester polymers after absorption of the liquid.

In contrast, the collagen film containing 100 wt % is more fully cross-linked. However, because the hexafluoroisopropanol used as the solvent in the film preparation can make the film contain both natural collagen and denatured collagen (refer to FIG. 1A and FIG. 1B), 100 wt % collagen film immersed in a liquid still has swelling properties, but its swelling proportion is lower than those of the films containing 70-90 wt % collagen.

With regard to the film containing 50 wt % collagen, since the polyester polymer content of the film accounts for half of the total film, it has a higher degree of influence on the film properties. The polyester polymer is a hydrophobic material and does not absorb water and swell, and thus the film containing 50 wt % collagen only shows a slight increase in size after 7 days (168 hours) of immersion in the liquid.

(2) With Regard to the Films Prepared in Preparation Examples 4, 5 and 6

According to the diameters of the non-fibrous film prepared by Preparation example 4 (80 wt % collagen and 20 wt % polycarprolactone, crosslinking for 1.5 hours), the non-fibrous film prepared by Preparation example 5 (80 wt % collagen and 20 wt % polycarprolactone, crosslinking for 2 hours) and the non-fibrous film prepared by Preparation example 6 (80 wt % collagen and 20 wt % polycarprolactone, crosslinking for 3 hours) in DMEM solution containing 10% fetal bovine serums determined at the time point 14 days and the time point 60 minutes, the swelling rates and swelling proportion per unit time of the respective films were calculated.

Swelling proportion per unit time is calculated based on the formula shown below.

Swelling proportion per unit time (%/hour)=swelling rate/film diameter at the time point 60 minutes*100

Film diameter at the time point 60 minutes: initial diameter of the film after immersion of the film in an aqueous liquid

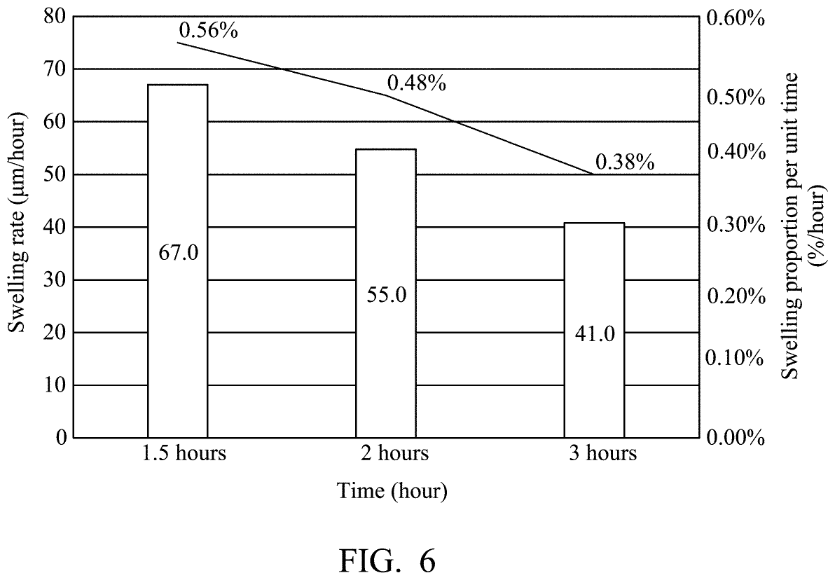

The results are shown in FIG. 6

FIG. 6 shows that the swelling rates of the films subjected to different cross-linking treatments with different time are different, and the swelling rate is higher for films with shorter cross-linking time. Therefore, it is known that the swelling rate of the film can be adjusted by the time of crosslinking for the film.

(3) With Regard to the Films Prepared in Preparation Examples 9 and 10

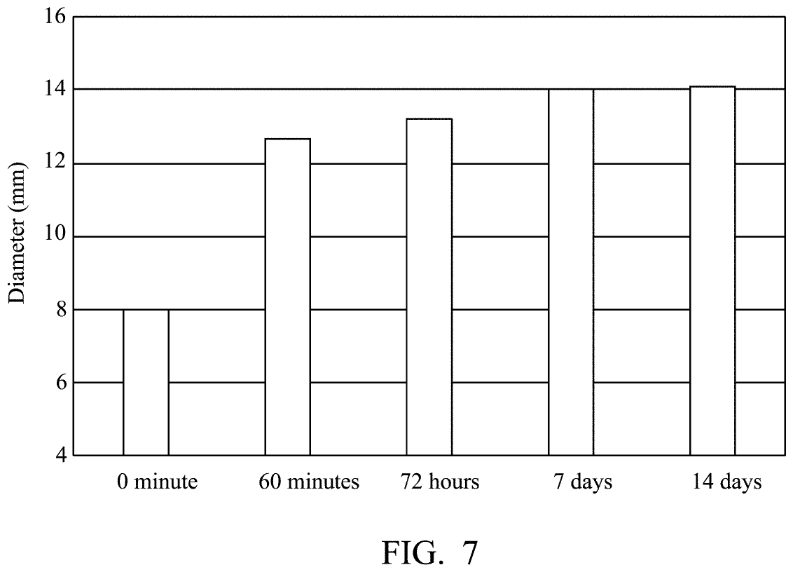

The film diameters of the non-fibrous film prepared in Preparation example 9 (90 wt % collagen and 10 wt % polylactic acid) determined in PBS buffer at different time points are as shown in FIG. 7.

Compared to at the time point 1 hour, the swelling proportions of the non-fibrous film prepared in Preparation example 9 (90 wt % collagen and 10 wt % polylactic acid) and the non-fibrous film prepared in Preparation example 10 (70 wt % collagen and 30 wt % polylactic acid) in PBS buffer at the time point 7 days and the time point 14 days are calculated, respectively. The results are shown in Table 5.

TABLE 5
Compared to at the time point 1 hour, the swelling proportions
of the non-fibrous film prepared in Preparation example
9 (90 wt % collagen and 10 wt % polylactic acid) and the
non-fibrous film prepared in Preparation example 10 (70
wt % collagen and 30 wt % polylactic acid) in PBS buffer
at the time point 7 days and the time point 14 days.

| | | Swelling |
|---|---|---|
| | | proportions |
| | | (%) |
| | Time | PBS |
| The film prepared in Preparation example 9 | 7 days | 10.41 |
| (90 wt % collagen and 10 wt % polylactic acid) | 14 days | 11.37 |
| The film prepared in Preparation example 10 | 7 days | 0.75 |
| (70 wt % collagen and 30 wt % polylactic acid) | 14 days | −0.87 |

According to FIG. 7 and Table 5, it is known that the film sizes of the collagen films containing polylactic acid in the aqueous liquid also increase with time.

(4) With Regard to the Films Prepared in Comparative Preparation Example 3 and

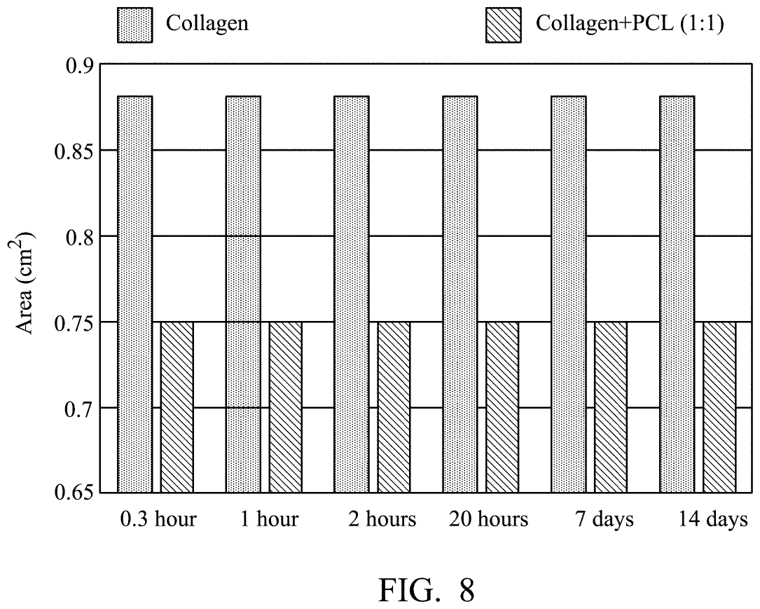

The areas of the electrospun films (100 wt % collagen) prepared in Comparative preparation example 3 and the electrospun films (50 wt % collagen and 50 wt % polycarprolactone) prepared in Comparative preparation example 4 determined in PBS buffer at different time points are shown in FIG. 8.

Based on FIG. 8, it is known that the electrospun films have no swelling effect.

### Example 5

Cell Culture Test

The films prepared in the Preparation example and the Comparative preparation example were subjected to cell culture tests, respectively.

1. Methods

Fibroblasts (HSF p′7) were cultured in DMEM high glucose medium containing 10% fetal bovine serum and penicillin-streptomycin (100 U/mL).

The film to be tested was immersed in 75% ethanol for 15 minutes, and then the ethanol was removed and allowed to evaporate from the film to dry. When the drying step was completed, the sterilization of the film to be tested is completed.

Next, the films were immersed in sterile water for 40 minutes and then immersed in cell culture medium for 15 minutes. After that, the films were placed into the holes of a 12-hole plate and fixed with plastic rings. The fibroblasts (HSF p′7) were inoculated into the holes with the films of the 12-hole plate mentioned above, at an inoculation density of 50,000 cells/cm². The fibroblasts (HSF p′7) were inoculated at the same inoculation density in the holes without film of the 12-well plate mentioned above as a control group. After the on the next day, after the cells attached, the plastic rings for fixation were removed.

Changes of cells on the films were observed through PKH67 live cell staining. The cell morphology was photographed with a fluorescent microscope on Day 1, 3, 7 and 14 and the changes in material sizes were determined.

2. Results

(1) With Regard to the Films Prepared in Preparation Example 3

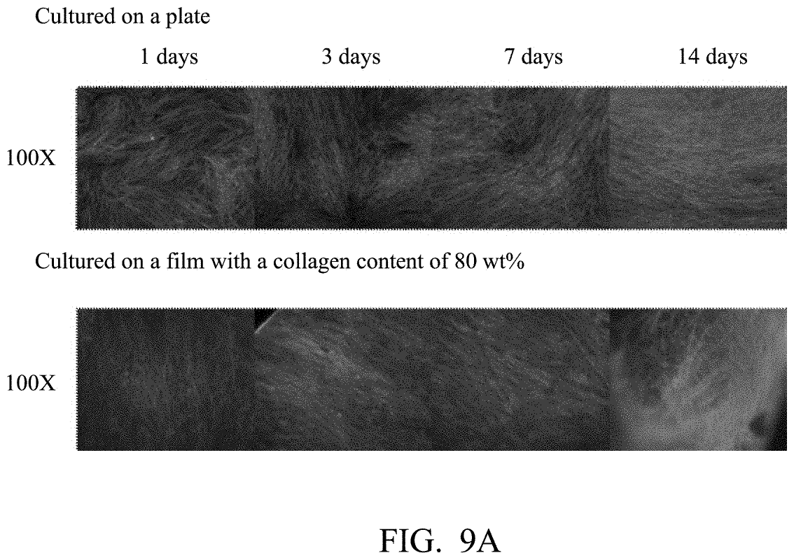

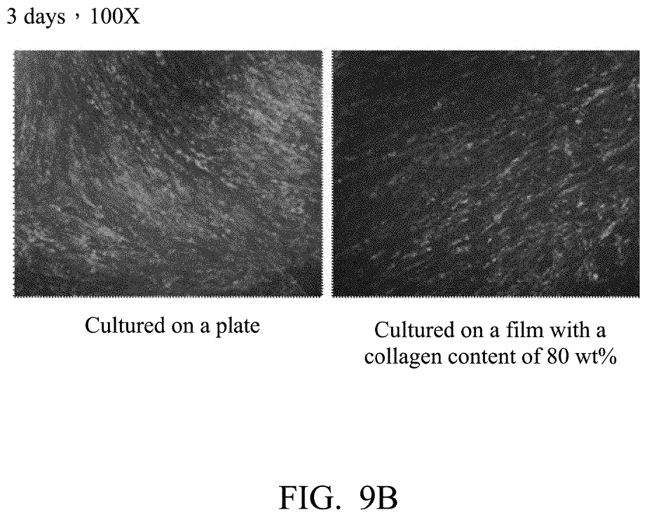

The results of the cell culture test of the non-fibrous film prepared in prepared in Preparation example 3 are shown in FIG. 9A and FIG. 9B.

FIG. 9A shows photographs of the growth status of cells on the non-fibrous film prepared in prepared in Preparation example 3 (80 wt % collagen and 20 wt % polycarprolactone) and cells on the culture plate on Day 1, 3, 7 and 14 in the cell culture test.

FIG. 9B shows photographs of the growth status of cells on the non-fibrous film prepared in prepared in Preparation example 3 (80 wt % collagen and 20 wt % polycarprolactone) and on the culture plate on Day 3 in the cell culture test.

According to FIG. 9A and FIG. 9B, it is known that the cells on the film of the present disclosure grow and arrange in the same direction. In the cell culture medium, the size of the film continuously increase over time, and the increase in the size of the film causes a physical mechanical stress on the cells to allow the cells show a property of growing in the same direction. In contrast, the growth status of cells on the culture plate is more disorganized.

(2) With Regard to the Film Prepared in Comparative Preparation Example 2

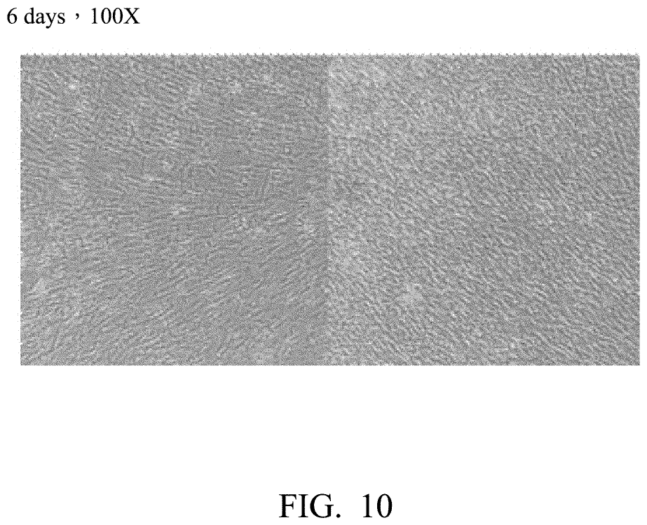

The result of the cell culture test of the non-fibrous film prepared in Comparative preparation example 2 (90 wt % collagen and 10 wt % polycarprolactone (acidic aqueous solution as a solvent)) is shown in FIG. 10.

According to FIG. 10, it is known that similar to the growth status of cells on culture plate, the growth status of cells on the non-fibrous film prepared in Comparative preparation example 2 (90 wt % collagen and 10 wt % polycarprolactone (acidic aqueous solution as a solvent)) is more disorganized and only occasionally arranged in a directional manner.

Based on the foregoing, it is known that, during cell culture, since the film of the present disclosure have changes in size, it provides the cells grow thereon with a physical mechanical stress and a stimulus of structural changes on the surface to which the cells are attached to allow the cells grow in the same direction and to promote cell differentiation.

It will be apparent to those skilled in the art that various modifications and variations can be made to the disclosed embodiments. It is intended that the specification and examples be considered as exemplary only, with a true scope of the disclosure being indicated by the following claims and their equivalents.

1. A non-fibrous film, of which the composition comprises:
a collagen; and
a polyester polymer,
wherein a content of the polyester polymer in the non-fibrous film is 1-60 wt %, and
wherein the non-fibrous film has a swelling rate of 1-200 μm/hour or a swelling proportion per unit time of 0.1-2%/hour in an aqueous liquid.

2. The non-fibrous film as claimed in claim 1, wherein the collagen comprises at least one of the following:
type I collagen, type II collagen and type III collagen.

3. The non-fibrous film as claimed in claim 1, wherein the collagen is type I collagen.

4. The non-fibrous film as claimed in claim 1, wherein a content of the polyester polymer in the non-fibrous film is 1-50 wt %.

5. The non-fibrous film as claimed in claim 1, wherein the composition of the non-fibrous film consists of the collagen and the polyester polymer, and a weight ratio of the collagen to the polyester polymer is 95:5, 90:10, 80:20, 70:30 or 50:50.

6. The non-fibrous film as claimed in claim 1, wherein a molecular weight of the polyester polymer is 50,000-1,500,000 Da.

7. The non-fibrous film as claimed in claim 1, wherein an intrinsic viscosity of the polyester polymer is 0.15-7.0 dl/g.

8. The non-fibrous film as claimed in claim 1, wherein the polyester polymer comprises at least one of the following:
polycarprolactone (PCL), polylactic acid (PLA), polyglycolic acid (PGA), polyhydroxybutyrate (PHB) and polyhydroxyvalerate (PHV).

9. The non-fibrous film as claimed in claim 1, wherein the non-fibrous film is a porous film.

10. The non-fibrous film as claimed in claim 9, wherein a pore size of the porous film is 1-50 μm.

11. The non-fibrous film as claimed in claim 9, wherein the polyester polymer is polycarprolactone, wherein a molecular weight of the polycarprolactone is 800,000-1,500,000 Da.

12. The non-fibrous film as claimed in claim 1, wherein the non-fibrous film is a non-porous film.

13. The non-fibrous film as claimed in claim 12, wherein the polyester polymer is polylactic acid, wherein an intrinsic viscosity of the polylactic acid is 0.15-7.0 dl/g.

14. A non-fibrous film, which is prepared by a method,
wherein the method comprises:
(a) preparing a mixture solution,
a solute of which comprises:
a collagen and a polyester polymer,
wherein a content of the polyester polymer in the non-fibrous film is 1-60 wt %, and
a solvent of which comprises a perfluorocarbon solvent; and
(b) drying the mixture solution to form a film,
wherein the film has a swelling rate of 1-200 μm/hour or a swelling proportion per unit time of 0.1-2%/hour in an aqueous liquid.

15. The non-fibrous film as claimed in claim 14, wherein a concentration of the solute in the mixture solution is 0.5-20 wt %.

16. The non-fibrous film as claimed in claim 14, wherein the collagen comprises at least one of the following:
type I collagen, type II collagen and type III collagen.

17. The non-fibrous film as claimed in claim 14, wherein the collagen is type I collagen.

18. The non-fibrous film as claimed in claim 14, wherein a content of the polyester polymer in the non-fibrous film is 1-50 wt %.

19. The non-fibrous film as claimed in claim 14, wherein the composition of the non-fibrous film consists of the collagen and the polyester polymer, and a weight ratio of the collagen to the polyester polymer is 95:5, 90:10, 80:20, 70:30 or 50:50.

20. The non-fibrous film as claimed in claim 14, wherein a molecular weight of the polyester polymer is 50,000-1,500,000 Da.

21. The non-fibrous film as claimed in claim 14, wherein an intrinsic viscosity of the polyester polymer is 0.15-7.0 dl/g.

22. The non-fibrous film as claimed in claim 14, wherein the polyester polymer comprises at least one of the following:
polycarprolactone, polylactic acid, polyglycolic acid, polyhydroxybutyrate and polyhydroxyvalerate.

23. The non-fibrous film as claimed in claim 14, wherein the non-fibrous film is a porous film.

24. The non-fibrous film as claimed in claim 23, wherein a pore size of the porous film is 1-50 μm.

25. The non-fibrous film as claimed in claim 23, wherein the polyester polymer is polycarprolactone, wherein a molecular weight of the polycarprolactone is 50,000-200,000 Da.

26. The non-fibrous film as claimed in claim 14, wherein the non-fibrous film is a non-porous film.

27. The non-fibrous film as claimed in claim 26, wherein the polyester polymer is polylactic acid, wherein an intrinsic viscosity of the polylactic acid is 0.15-7.0 dl/g.

28. The non-fibrous film as claimed in claim 14, wherein the perfluorocarbon solvent comprises hexafluoroisopropanol, hexafluoroacetone, 1,1,1-trichlorotrifluoroacetone, trifluoroacetic acid, perfluoroperhydrophenanthrene or perfluoro(methylcyclohexane).

29. The non-fibrous film as claimed in claim 14, wherein the aqueous liquid comprises water, buffer or medium.

30. The non-fibrous film as claimed in claim 14, wherein the method further comprises after the step (b), performing a crosslinking treatment on the film.

31. The non-fibrous film as claimed in claim 30, wherein the time for the crosslinking treatment is 0.1-10 hours.

32. The non-fibrous film as claimed in claim 30, wherein the crosslinking treatment is performed by a cross-linking agent, and the cross-linking agent comprises formaldehyde, glutaraldehyde, glyoxal, malondialdehyde, succinyl dialdehyde, phthalaldehyde, dialdehyde starch, polyacrolein or polymethacrolein.

33. A cell sheet, comprising:
the non-fibrous film as claimed in claim 1; and
at least one cell layer on the non-fibrous film,
wherein the cell layer is composed of a plurality of cells, and the plurality of cells are arranged in the same direction.

34. The cell sheet as claimed in claim 33, wherein the cells comprise fibroblast, myoblasts, epithelial cells, endothelial cells, progenitor cells, tenocyte, stem cells, mesenchymal stem cells, bone marrow stem cells or adipose derived stem cells.
